(19) United States
(12) Patent Application Publication
(10) Pub. No.: US 2026/0325361 A1
(43) Pub. Date: Oct. 22, 2026

(54) SKIN PATCH SHEET

(71) Applicant: TOPPAN Holdings Inc., Tokyo (JP)

(72) Inventors: Saeko NOMURA, Tokyo (JP); Natsumi MIYAUCHI, Tokyo (JP); Junya TANABE, Tokyo (JP)

(73) Assignee: TOPPAN Holdings Inc., Tokyo (JP)

(21) Appl. No.: 19/463,102

(22) Filed: Jan. 29, 2026

Related U.S. Application Data
(63) Continuation of application No. PCT/JP2024/025854, filed on Jul. 18, 2024.

(30) Foreign Application Priority Data
Aug. 17, 2023 (JP) 2023-132804

(51) Int. Cl. A61F 13/02

(52) U.S. Cl. CPC A61F 13/0259; A61F 13/0243

(57) ABSTRACT

A region of a carrier sheet that does not overlap a patch tape is a first region, and a region that overlaps the patch tape is a second region. The carrier sheet has a cut extending beyond the boundary between the first region and the second region from a portion of the outer edge of the carrier sheet included in the first region toward the patch tape when viewed from a perspective facing a plane on which the carrier sheet extends. The 180° peeling strength according to JIS Z 0237:2009 between the carrier sheet and the substrate is 2 N/25 mm or less. The carrier sheet has a thickness of 10 μm or more and 400 μm or less. When the thickness of the carrier sheet 11 is T, a depth D1 of the cut satisfies the following formula: ¼T≤D1≤T.

### CROSS-REFERENCE TO RELATED APPLICATIONS

The present application is a bypass continuation of International Application No. PCT/JP2024/025854, filed Jul. 18, 2024, which claims priority to Japanese Patent Application No. 2023-132804, filed Aug. 17, 2023, the entire contents of each are incorporated herein by reference in their entirety.

### TECHNICAL FIELD

The present disclosure relates to a skin patch sheet.

### BACKGROUND ART

Patch sheets for the skin are widely used for the purpose of protecting affected areas of the skin from the outside or hiding part of the skin from the outside. Such a patch sheet includes a patch tape held between a carrier sheet and a release sheet. The patch tape includes a substrate in contact with the carrier sheet, and an adhesive layer in contact with the release sheet. When using the patch sheet, the release sheet is first peed from the adhesive layer. Then, the laminate of the patch tape and the carrier sheet is attached to the skin by the adhesive layer. Subsequently, the carrier sheet is peeled from the substrate (see, for example, PTL 1).

### CITATION LIST

### Patent Literature

PTL 1: JP 6706398 B

### SUMMARY OF THE INVENTION

### Technical Problem

The substrate to be attached to the skin is required to have high tensile elongation at break, similar to polyurethane, with the aim to achieve conformability to the skin. On the other hand, such a substrate also has high adhesion to a polyolefin-based carrier sheet and a polyester-based carrier sheet, making it difficult to peel the carrier sheet from the substrate attached to the skin.

### Solution to Problem

An aspect of a skin patch sheet includes a carrier sheet, a release sheet, and a patch tape located between the carrier sheet and the release sheet. The patch tape includes a substrate that is in contact with the carrier sheet and has higher tensile elongation at break than that of the carrier sheet and the release sheet, and an adhesive layer that is in contact with the release sheet and is to be attached to the skin after the release sheet is peeled from the adhesive layer. The area of the carrier sheet is larger than the area of the patch tape. The carrier sheet includes a first region that does not overlap the patch tape and a second region that overlaps the patch tape. The carrier sheet has a cut extending beyond the boundary between the first region and the second region from a portion of the outer edge of the carrier sheet included in the first region toward the patch tape, when viewed from a perspective facing a plane on which the carrier sheet extends. The 180° peeling strength according to JIS Z 0237:2009 between the carrier sheet and the substrate is 2 N/25 mm or less. The carrier sheet has a thickness of 10 μm or more and 400 μm or less. When the thickness of the carrier sheet is T, a depth D1 of the cut satisfies the following formula:

¼T≤D1≤T

An aspect of a skin patch sheet includes a carrier sheet, a release sheet, and a patch tape located between the carrier sheet and the release sheet. The patch tape includes a substrate that is in contact with the carrier sheet and has higher tensile elongation at break than that of the carrier sheet and the release sheet, and an adhesive layer that is in contact with the release sheet and is to be attached to the skin after the release sheet is peeled from the adhesive layer. The area of the carrier sheet is larger than the area of the patch tape. The carrier sheet includes a first region that does not overlap the patch tape and a second region that overlaps the patch tape. The carrier sheet has a cut extending beyond the boundary between the first region and the second region from a portion of the outer edge of the carrier sheet included in the first region toward the patch tape, when viewed from a perspective facing a plane on which the carrier sheet extends. The 180° peeling strength according to JIS Z 0237:2009 between the carrier sheet and the substrate is 2 N/25 mm or less. The carrier sheet has a trouser tear strength according to JIS K 7128-1:1998 of 10 N/mm or less. When the thickness of the carrier sheet is T, a remaining thickness TR1 of the carrier sheet connected to the cut in a thickness direction of the carrier sheet satisfies the following formula:

0≤TR1≤¾T.

### BRIEF DESCRIPTION OF THE DRAWINGS

FIG. 1 is a cross-sectional view showing the structure of a skin patch sheet.

FIG. 2 is a plan view showing the structure of the skin patch sheet shown in FIG. 1.

FIG. 3 is a cross-sectional view showing a portion of the carrier sheet and a portion of the substrate in the skin patch sheet shown in FIG. 1.

FIG. 4 is a plan view showing the structure of a first modification of the skin patch sheet.

FIG. 5 is a plan view showing the structure of a second modification of the skin patch sheet.

FIG. 6 is a plan view showing the structure of a third modification of the skin patch sheet.

FIG. 7 is a plan view showing the structure of a fourth modification of the skin patch sheet.

FIG. 8 is a plan view showing the structure of a fifth modification of the skin patch sheet.

FIG. 9 is a plan view showing the structure of a sixth modification of the skin patch sheet.

FIG. 10 is a plan view showing the structure of a seventh modification of the skin patch sheet.

FIG. 11 is a plan view showing the structure of an eighth modification of the skin patch sheet.

FIG. 12 is a plan view showing the structure of a ninth modification of the skin patch sheet.

FIG. 13 is a plan view showing the structure of a tenth modification of the skin patch sheet.

FIG. 14 is a plan view showing the structure of an eleventh modification of the skin patch sheet.

FIG. 15 is a table showing the evaluation results in the skin patch sheets of the Examples and Comparative Examples.

### DESCRIPTION OF THE EMBODIMENTS

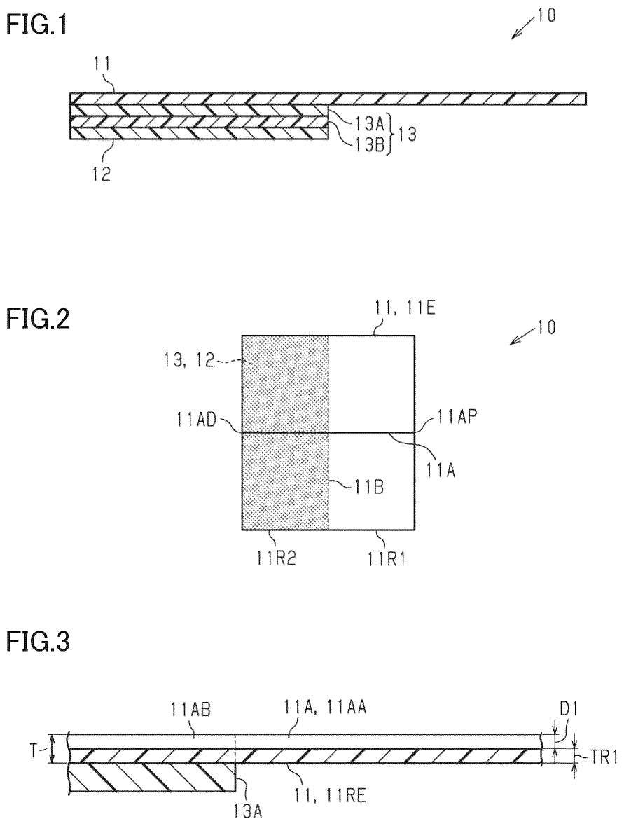

An embodiment of a skin patch sheet will be described with reference to FIGS. 1 to 15.

### Skin Patch Sheet

First and second configurations of the skin patch sheet of the present disclosure will be described with reference to FIGS. 1 to 3. In the following, after the common points between the first and second configurations are described, the first and second configurations will be described in order.

As shown in FIG. 1, a skin patch sheet 10 includes a carrier sheet 11, a release sheet 12, and a patch tape 13 located between the carrier sheet 11 and the release sheet 12. The patch tape 13 includes a substrate 13A and an adhesive layer 13B. The substrate 13A is in contact with the carrier sheet 11 and has higher tensile elongation at break than that of the carrier sheet 11 and the release sheet 12. The adhesive layer 13B is in contact with the release sheet 12. The adhesive layer 13B is attached to the skin after the release sheet 12 is peeled from the adhesive layer 13B.

The 180° peeling strength according to JIS Z 0237:2009 between the carrier sheet 11 and the substrate 13A is 2 N/25 mm or less. The 180° peeling strength is a value measured by a method according to JIS Z 0237:2009 “Testing methods of pressure-sensitive adhesive tapes and sheets.”

As shown in FIG. 2, the area of the carrier sheet 11 is larger than the area of the patch tape 13. When viewed from a perspective facing a plane on which the skin patch sheet 10 extends, the total area of the carrier sheet 11 is larger than the total area of the patch tape 13. A region of the carrier sheet 11 that does not overlap the patch tape 13 is a first region 11R1, and a region that overlaps the patch tape 13 is a second region 11R2. The carrier sheet 11 is composed of the first region 11R1 and the second region 11R2, and the sum of the area of the first region 11R1 and the area of the second region 11R2 is larger than the area of the patch tape 13. In the example shown in FIG. 2, the release sheet 12 has the same shape as the patch tape 13.

The carrier sheet 11 has a cut 11A. In the example shown in FIG. 2, the carrier sheet 11 has only one cut 11A. The cut 11A extends beyond the boundary 11B between the first region 11R1 and the second region 11R2 from a portion of the outer edge 11E of the carrier sheet 11 included in the first region 11R1 toward the patch tape 13 when viewed from a perspective facing a plane on which the carrier sheet 11 extends.

The cut 11A includes a base end 11AP and a tip 11AD. The base end 11AP of the cut 11A is located in a portion of the outer edge 11E of the carrier sheet 11 included in the first region 11R1. In contrast, the tip 11AD of the cut 11A is located in a portion of the outer edge 11E of the carrier sheet 11 included in the second region 11R2. The tip 11AD of the cut 11A may be located within a region of the second region 11R2 of the carrier sheet 11 inside the outer edge 11E.

That is, in the example shown in FIG. 2, the carrier sheet 11 has a rectangular shape, and the first region 11R1 and the second region 11R2 are arranged along the short-side direction. The cut 11A has a length across the entire carrier sheet 11 in the short-side direction. The cut 11A may have a length extending across the entire first region 11R1 and partially into the second region 11R2 in the short-side direction of the carrier sheet 11.

As shown in FIG. 2, the cut 11A may have a linear shape. Alternatively, the cut 11A may have a curved or broken-line shape. When the cut 11A has a broken-line shape, the cut 11A may have only one bent part or two or more bent parts.

FIG. 3 is a cross-sectional view including a portion of the carrier sheet 11 and a portion of the substrate 13A. FIG. 3 is also a cross-sectional view along a plane including the cut 11A of the carrier sheet 11.

In the example shown in FIG. 3, the cut 11A has a depth D1 that reaches only a portion of the carrier sheet 11 in the thickness direction. The cut 11A may have a depth D1 across the entire carrier sheet 11 in the thickness direction. When the cut 11A has a depth D1 that reaches only a portion of the carrier sheet 11 in the thickness direction, the carrier sheet 11 has a remaining part 11RE connected to the cut 11A in the thickness direction of the carrier sheet 11. The remaining part 11RE of the carrier sheet 11 has a remaining thickness TR1 that is obtained by subtracting the depth D1 of the cut 11A from the thickness T of the carrier sheet 11.

The cut 11A is composed of a first portion 11AA and a second portion 11AB. The first portion 11AA is a portion of the cut 11A located in the first region 11R1 of the carrier sheet 11. The second portion 11AB is a portion of the cut 11A located in the second region 11R2 of the carrier sheet 11. In the example shown in FIG. 3, the depth of the first portion 11AA is equal to the depth of the second portion 11AB. The depth of the first portion 11AA may be different from the depth of the second portion 11AB.

### First Configuration

The first configuration of the skin patch sheet 10 satisfies the following conditions.
(Condition 1-1) The carrier sheet 11 has a thickness of 10 μm or more and 400 μm or less.
(Condition 1-2) When the thickness of the carrier sheet 11 is T, the depth D1 of the cut 11A satisfies the following formula:

¼T≤D1≤T

In the carrier sheet 11 having a thickness of 10 μm or more and 400 μm or less, the 180° peeling strength is 2 N/25 mm or less, and the depth D1 of the cut 11A extending from the outside of the patch tape 13 to the inside of the patch tape 13 is included within the range of ¼T or more and T or less. Therefore, it is easy to peel the carrier sheet 11 from substrate 13A starting from the cut 11A. In the present disclosure, the adhesion strength between the carrier sheet 11 and the substrate 13A is evaluated using the 180° peeling strength.

From the viewpoint of facilitating the peeling of the carrier sheet 11 starting from the cut 11A, the depth D1 of the cut 11A is preferably deep. Therefore, the lower limit value of the depth D1 may be ½T or ¾T. From the viewpoint of preventing the substrate 13A from being cut during the production of the skin patch sheet 10, and from the viewpoint of preventing foreign substances from reaching the substrate 13A through the cut 11A, the depth D1 of the cut 11A is preferably shallow. Therefore, the depth D1 may be less than T. The upper limit value of the depth D1 may be ¾T or ½T.

That is, from the viewpoint of facilitating peeling, the depth D1 of the cut 11A may be, for example, ½T or more and T or less, or ¾T or more and T or less. From the viewpoint of preventing the substrate 13A from being cut, and from the viewpoint of preventing the arrival of foreign substances, the depth D1 of the cut 11A may be, for example, ¼T or more and less than T, ¼T or more and ¾T or less, or ¼T or more and ½T or less. From the viewpoint of achieving the facilitation of peeling, the prevention of the substrate 13A from being cut, and the prevention of the arrival of foreign substances, the depth of the cut 11A may be, for example, ½T or more and less than T, or ½T or more and ¾T or less.

The skin patch sheet 10 may also satisfy the following conditions 1-3 and 1-4.
(Condition 1-3) The depth of a portion of the cut 11A that overlaps the patch tape 13 in the carrier sheet 11 is shallower than the depth of a portion that does not overlap the patch tape 13 in the carrier sheet 11. That is, the depth of the first portion 11AA of the cut 11A is deeper than the depth of the second portion 11AB of the cut 11A.

A portion of the cut 11A located on the patch tape 13 does not penetrate the carrier sheet 11. Therefore, during the production of the skin patch sheet 10, the carrier sheet 11 and the substrate 13A can be prevented from being cut, and foreign substances can be prevented from reaching the substrate through the cut 11A before using the skin patch sheet 10.
(Condition 1-4) The carrier sheet 11 has a trouser tear strength according to JIS K 7128-1:1998 of 10 N/mm or less.

Since the trouser tear strength is 10 N/mm or less, while the tearability of the carrier sheet 11 is improved, the carrier sheet 11 and the substrate 13A can be prevented from being cut during the production of the skin patch sheet 10. In addition, foreign substances can be prevented from reaching the substrate 13A through the cut 11A before using the skin patch sheet 10. The lower limit value of the trouser tear strength may be greater than 0, for example, 0.01 N/mm.

When the skin patch sheet 10 satisfies the condition 1-4, the depth of the first portion 11AA may be equal to the thickness T, whereas the depth of the second portion 11AB may be less than the thickness T. Alternatively, when the skin patch sheet 10 satisfies the condition 1-4, the depth of the first portion 11AA and the depth of the second portion 11AB both may be less than the thickness T. From the viewpoint of facilitating the peeling of the carrier sheet 11 starting from the cut 11A, the depth of the first portion 11AA is preferably equal to the thickness T.

### Second Configuration

The second configuration of the skin patch sheet 10 satisfies the following conditions.
(Condition 2-1) The carrier sheet 11 has a trouser tear strength according to JIS K 7128-1:1998 of 10 N/mm or less.
(Condition 2-2) When the thickness of the carrier sheet 11 is T, the remaining thickness TR1, which is the thickness of the remaining part 11RE of the carrier sheet 11 connected to the cut 11A, in the thickness direction of the carrier sheet 11 satisfies the following formula:

0≤TR1≤¾T.

In the carrier sheet 11 that has a trouser tear strength of 10 N/mm or less, the 180° peeling strength is 2 N/mm or less, and the remaining thickness of the carrier sheet 11 in the cut 11A extending from the outside of the patch tape 13 to the inside of the patch tape 13 is included within the range of 0 or more and ¾T or less. Therefore, it is easy to peel the carrier sheet 11 from substrate 13A starting from the cut 11A.

From the viewpoint of facilitating the peeling of the carrier sheet 11 starting from the cut 11A, the remaining thickness TR1 in the cut 11A is preferably thin. Therefore, the upper limit value of the remaining thickness TR1 may be ½T or ¼T. From the viewpoint of preventing the substrate 13A from being cut during the production of the skin patch sheet 10, and from the viewpoint of preventing foreign substances from reaching the substrate 13A through the cut 11A, the remaining thickness TR1 in the cut 11A is preferably thick. Therefore, the remaining thickness TR1 may be more than 0. In addition, the upper limit value of the remaining thickness TR1 may be ¼T or ½T.

That is, from the viewpoint of facilitating peeling, the remaining thickness TR1 may be 0 or more and ½T or less, or 0 or more and ¼T or less. From the viewpoint of preventing the substrate 13A from being cut, and from the viewpoint of preventing the arrival of foreign substances, the remaining thickness TR1 may be more than 0 and ¾ or less, ¼T or more and ¾T or less, or ½T or more and ¾T or less. From the viewpoint of achieving the facilitation of peeling, the prevention of the substrate 13A from being cut, and the prevention of the arrival of foreign substances, the remaining thickness TR1 may be, for example, more than 0 and ½T or less, or ¼T or more and ½T or less.

The skin patch sheet 10 may also satisfy the following condition 2-3.
(Condition 2-3) The remaining thickness of a portion of the cut 11A that overlaps the patch tape 13 in the carrier sheet 11 is thinner than the remaining thickness of a portion that does not overlap the patch tape 13 in the carrier sheet 11. That is, the remaining thickness of the first portion 11AA of the cut 11A is thinner than the remaining thickness of the second portion 11AB of the cut 11A.

A portion of the cut 11A located on the patch tape 13 does not penetrate the carrier sheet 11. Therefore, during the production of the skin patch sheet 10, the carrier sheet and the substrate 13A can be prevented from being cut, and foreign substances can be prevented from reaching the substrate through the cut 11A before using the skin patch sheet 10.

When the skin patch sheet 10 satisfies the condition 2-3, the remaining thickness of the first portion 11AA may be 0, whereas the remaining thickness of the second portion 11AB may be more than 0. Alternatively, when the skin patch sheet 10 satisfies the condition 2-3, the remaining thickness of the first portion 11AA and the remaining thickness of the second portion 11AB both may be less than the thickness T. From the viewpoint of facilitating the peeling of the carrier sheet 11 starting from the cut 11A, the remaining thickness of the first portion 11AA is preferably 0.

Thus, either of the first and second configurations of the skin patch sheet 10 makes it easy to peel the carrier sheet 11 from the substrate 13A starting from the cut 11A. When the substrate 13A or the adhesive layer 13B contains an active ingredient for the skin, or when the skin patch sheet 10 is stored in an environment that satisfies at least one of high temperature and high humidity, the adhesion strength between the substrate 13A and the carrier sheet 11 may be further increased. The skin patch sheet 10 of the present disclosure makes it possible to easily peel the carrier sheet 11 from the substrate 13A even when the adhesion strength is further increased.

It is also possible to combine the first configuration and the second configuration. That is, the skin patch sheet satisfies the conditions 1-1 and 2-1, and the cut 11A satisfies both the conditions 1-2 and 2-2.

### Modifications

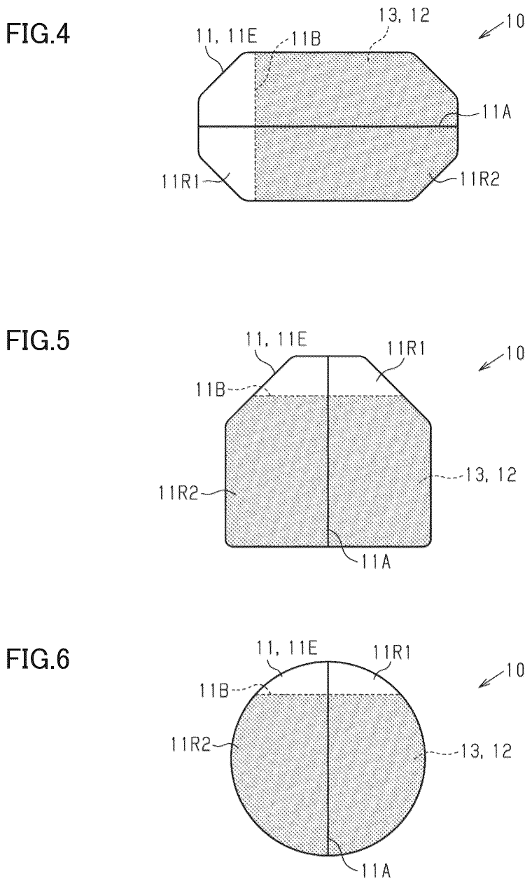

As shown in FIGS. 4 to 14, the first and second configurations of the skin patch sheet 10 described above can be carried out with the following modifications.

The outer shapes of the skin patch sheets 10 shown in FIGS. 4 to 9 are different from that of the skin patch sheet 10 described with reference to FIGS. 1 to 3. Further, in the skin patch sheet 10 shown in FIG. 9, the number of first regions 11R1 is different from that of the skin patch sheet 10 described with reference to FIGS. 1 to 3.

In the first modification shown in FIG. 4, the outer shape of the carrier sheet 11 is octagonal. The outer shape of the carrier sheet 11 is also a rectangular shape with each corner portion cut off. The cut 11A has a length across the entire carrier sheet 11 in the lengthwise direction. The outer shape of the patch tape 13 and the outer shape of the release sheet 12 are approximately rectangular. In the patch tape 13 and the release sheet 12, two adjacent corner portions of the rectangle in the widthwise direction are cut off.

In the second modification shown in FIG. 5, the outer shape of the carrier sheet 11 is hexagonal. The outer shape of the carrier sheet 11 is also a rectangle shape with two adjacent corner portions in the widthwise direction cut off. The cut 11A has a length across the entire carrier sheet 11 in the lengthwise direction. The outer shape of the patch tape 13 and the outer shape of the release sheet 12 are hexagonal. The outer shape of the patch tape 13 and the outer shape of the release sheet 12 are also a rectangle shape with two adjacent corner portions in the widthwise direction cut off.

In the third modification shown in FIG. 6, the outer shape of the carrier sheet 11 is circular. The cut 11A passes through the center of the carrier sheet 11 and has a length equal to the diameter of the carrier sheet 11. The outer shape of the patch tape 13 and the outer shape of the release sheet 12 are defined by a major arc concentric with the carrier sheet 11 and a chord connecting the both ends of the major arc.

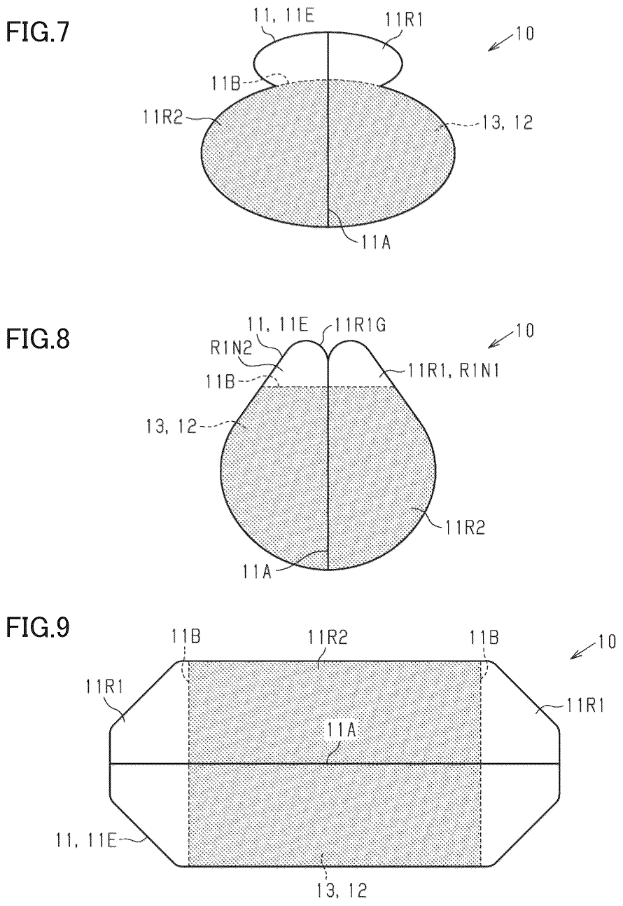

In the fourth modification shown in FIG. 7, the outer shape of the carrier sheet 11 is composed of a first elliptical arc and a second elliptical arc. The long axis in the second elliptical arc is longer than the long axis in the first elliptical arc. The first elliptical arc and the second elliptical arc are major arcs. The cut 11A has a length across the entire first region 11R1 and the entire second region 11R2. The outer shape of the patch tape 13 and the outer shape of the release sheet 12 are elliptical. The outer shape of the patch tape 13 and the outer shape of the release sheet 12 are concentric with the second elliptical arc. In the outer shape of the patch tape 13 and the outer shape of the release sheet 12, the length of the short axis and the length of the long axis are the same as those of the second elliptical arc. The first region 11R1 is surrounded by the first elliptical arc. The second region 11R2 is surrounded by the second elliptical arc.

In the fifth modification shown in FIG. 8, the outer shape of the carrier sheet 11 is substantially circular. The outer shape of the carrier sheet 11 is an approximately water droplet shape extending along the lengthwise direction. A first region 11R1 and a second region 11R2 are lined up in the lengthwise direction. The first region 11R1 has a groove 11R1G recessed toward the second region 11R2 at a position facing the boundary 11B between the first region 11R1 and the second region 11R2 in the lengthwise direction. The cut 11A has an end portion in the valley of the groove 11R1G and also has a full length in the lengthwise direction. The first region 11R1 is divided by the cut 11A into a first pinch portion R1N1 and a second pinch portion R1N2. The outer shape of the patch tape 13 and the outer shape of the release sheet 12 are defined by an approximately major arc segment included in the outer shape of the carrier sheet 11 and a straight line segment connecting the both ends of the arc segment and extending along the widthwise direction.

According to the fifth modification of the skin patch sheet 10, the user of the skin patch sheet 10 can pinch the first pinch portion R1N1 and the second pinch portion R1N2 sandwiching the groove 11R1G, and then pull the first pinch portion R1N1 and the second pinch portion R1N2 in a direction away from each other, thereby tearing the carrier sheet 11 along the cut 11A. Therefore, it becomes easy to tear the carrier sheet 11.

In the sixth modification shown in FIG. 9, the outer shape of the carrier sheet 11 is octagonal, as with the first modification of the skin patch sheet 10. The outer shape of the carrier sheet 11 is also a rectangular shape with each corner portion cut off. The cut 11A has a length across the entire carrier sheet 11 in the lengthwise direction. On the other hand, the outer shape of the patch tape 13 and the outer shape of the release sheet 12 are rectangular. As a result, neither end of the carrier sheet 11 overlaps the patch tape 13 in the lengthwise direction of the skin patch sheet 10. That is, the carrier sheet 11 includes two first regions 11R1 and one second region 11R2. In the lengthwise direction of the carrier sheet 11, the second region 11R2 is sandwiched between the two first regions 11R1. Since the second region 11R2 is sandwiched between the two first regions 11R1, it is easy to tear the carrier sheet 11 from both sides in the lengthwise direction along the cut 11A.

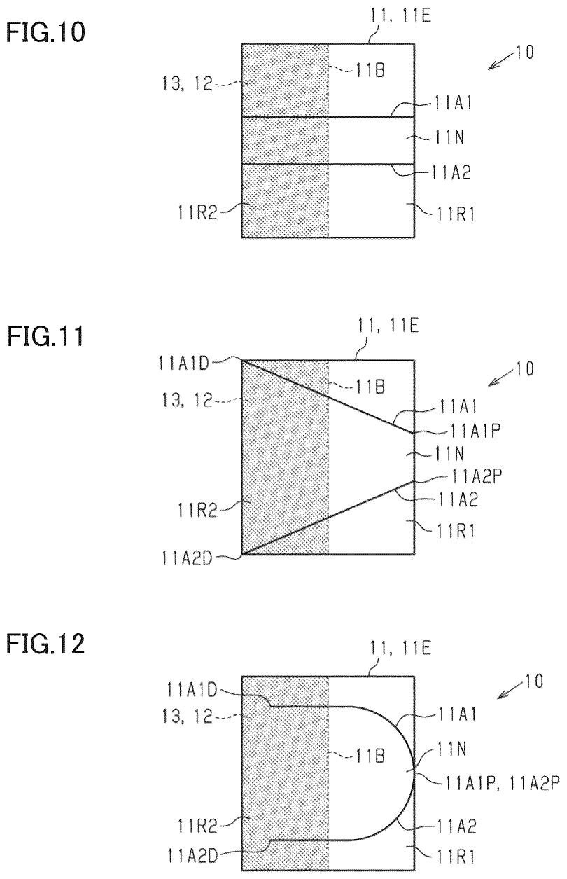

In the skin patch sheets 10 shown in FIGS. 10 to 14, the number of cuts 11A provided in the carrier sheet 11 is different from that of the skin patch sheet 10 described with reference to FIGS. 1 to 3. In the skin patch sheets 10 shown in FIGS. 10 to 14, the carrier sheet 11 has two cuts 11A. The number of cuts 11A provided in the carrier sheet 11 may be one or more, and may be two or three or more, as in the skin patch sheets 10 shown in FIGS. 10 to 14.

Of these, in the skin patch sheets 10 shown in FIGS. 10 to 13, the carrier sheet 11 includes a first cut 11A1 and a second cut 11A2. The first cut 11A1 has a configuration equivalent to that of the cut 11A of the first configuration or the second configuration described above. As with the first cut 11A1, the second cut 11A2 extends beyond the boundary 11B between the first region 11R1 and the second region 11R2 from a portion of the outer edge 11E of the carrier sheet 11 included in the first region 11R1 toward the patch tape 13 when viewed from a perspective facing a plane on which the carrier sheet 11 extends. As with the depth D1 of the first cut 11A1, when the thickness of the carrier sheet 11 is T, the depth D2 of the second cut 11A2 satisfies the following formula:

¼T≤D2≤T

When the thickness of the carrier sheet 11 is T, the remaining thickness TR2, which is the thickness of a remaining part of the carrier sheet 11 connected to the second cut 11A2, in the thickness direction of the second cut 11A2 may satisfy the following formula:

0≤TR2≤¾T

When viewed from a perspective facing a plane on which the carrier sheet 11 extends, the first cut 11A1 and the second cut 11A2 are spaced apart from each other along a first direction, and the cuts 11A1 and 11A2 each extend along a second direction that intersects with the first direction. The carrier sheet 11 includes a pinch portion 11N that is sandwiched between the first cut 11A1 and the second cut 11A2, and extends along the second direction.

Since it is possible for the user of the skin patch sheet 10 to hold the pinch portion 11N of the carrier sheet 11, it is easy to apply, to the carrier sheet 11, external force for peeling the carrier sheet 11 from the substrate 13A. With reference to the drawings, the structure of each modification will be described in more detail below. In the modifications shown in FIGS. 10 to 13, the outer shape of the carrier sheet 11 is rectangular.

In the seventh modification shown in FIG. 10, the first cut 11A1 and the second cut 11A2 both have a length across the entire skin patch sheet 10 in the widthwise direction. The first cut 11A1 and the second cut 11A2 are spaced apart from each other in the lengthwise direction of the skin patch sheet 10. The lengthwise direction is an example of the first direction. The first cut 11A1 is parallel to the second cut 11A2. The first cut 11A1 and the second cut 11A2 define a pinch portion 11N with a rectangular shape extending along the widthwise direction. The widthwise direction is an example of the second direction.

In the eighth modification shown in FIG. 11, the cuts 11A1 and 11A2 each extend along a direction that intersects with the widthwise direction. The first cut 11A1 extends along the widthwise direction, and is line-symmetric to the second cut 11A2 with respect to the axis of symmetry passing through the center of the carrier sheet 11 in the lengthwise direction. The distance between the first cut 11A1 and the second cut 11A2 in the lengthwise direction gradually becomes longer from the base end 11A1P of the first cut 11A1 toward the tip 11A1D. Therefore, the distance between the base end 11A1P of the first cut 11A1 and the distance between the base end 11A2P of the second cut 11A2 is the shortest, and the distance between the tip 11A1D of the first cut 11A1 and the tip 11A2D of the second cut 11A2 is the longest. The pinch portion 11N defined by the first cut 11A1 and the second cut 11A2 has a trapezoidal shape.

In the ninth modification shown in FIG. 12, the first cut 11A1 has a linear shape along the widthwise direction on the way from the tip 11A1D toward the base end 11A1P, and a shape curved to approach the second cut 11A2 on the way from the tip 11A1D toward the base end 11A1P. The second cut 11A2 has a linear shape along the widthwise direction on the way from the tip 11A2D toward the base end 11A2P, and a shape curved to approach the first cut 11A1 on the way from the tip 11A2D toward the base end 11A2P. The base end 11A2P of the second cut 11A2 is connected to the base end 11A1P of the first cut 11A1 in the outer edge 11E of the carrier sheet 11. The tips 11A1D and 11A2D of the respective cuts 11A1 and 11A2 are located within the second region 11R2.

The first cut 11A1 extends along the widthwise direction, and is line-symmetric to the second cut 11A2 with respect to the axis of symmetry passing through the center of the carrier sheet 11 in the lengthwise direction. The shape formed by the first cut 11A1 and the second cut 11A2 is U-shaped. The distance between the first cut 11A1 and the second cut 11A2 gradually becomes longer and then becomes constant in the direction from the base end 11A1P of the first cut 11A1 toward the tip 11A1D.

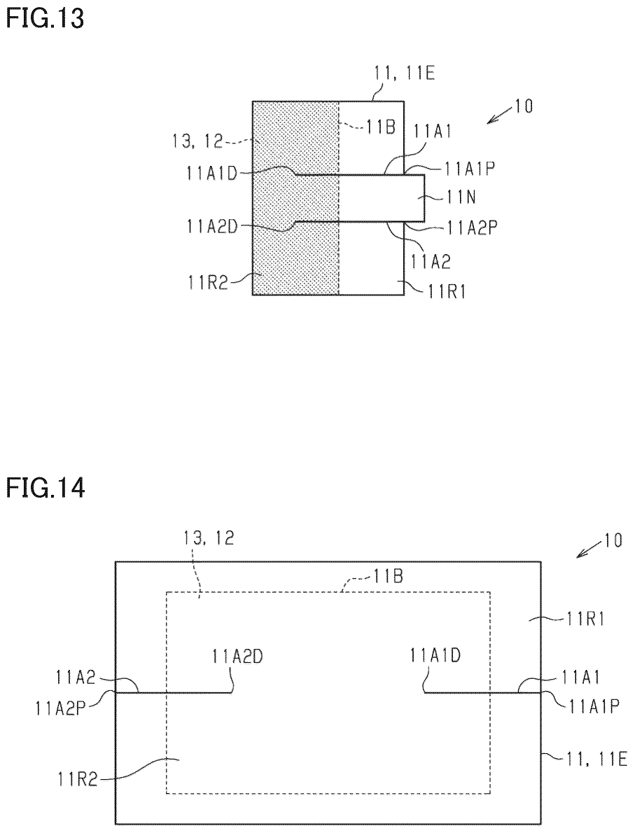

In the tenth modification shown in FIG. 13, each of the cuts 11A1 and 11A2 extends along the widthwise direction. The tips 11A1D and 11A2D of the respective cuts 11A1 and 11A2 are located within the second region 11R2. The pinch portion 11N is composed of a portion sandwiched between the first cut 11A1 and the second cut 11A2, and a portion located outside the cuts 11A1 and 11A2. In other words, the pinch portion 11N includes a portion more protruding than the base ends 11A1P and 11A2P towards the opposite side of the tips 11A1D and 11A2D with respect to the base ends 11A1P and 11A2P.

In each modification, from the viewpoint of making it easier to pinch the pinch portion 11N, the depths D1 and D2 of the portions of the cuts 11A1 and 11A2 located in the first region 11R1 are preferably equal to the thickness T of the carrier sheet 11. That is, the remaining thicknesses TR1 and TR2 of the respective cuts 11A1 and 11A2 are preferably 0.

In the eleventh modification of the skin patch sheet 10 shown in FIG. 14, the carrier sheet 11 includes a first cut 11A1 and a second cut 11A2. The first cut 11A1 has a configuration equivalent to that of the cut 11A of the first configuration or the second configuration described above. As with the first cut 11A1, the second cut 11A2 extends beyond the boundary 11B between the first region 11R1 and the second region 11R2 from a portion of the outer edge 11E of the carrier sheet 11 included in the first region 11R1 toward the patch tape 13 when viewed from a perspective facing a plane on which the carrier sheet 11 extends.

As with the depth D1 of the first cut 11A1, when the thickness of the carrier sheet 11 is T, the depth D2 of the second cut 11A2 satisfies the following formula: The base ends 11A1P and 11A2P of the respective cuts 11A1 and 11A2 located at the outer edge 11E of the carrier sheet 11 face each other across the patch tape.

¼T≤D2≤T

When the thickness of the carrier sheet 11 is T, the remaining thickness TR2, which is the thickness of a remaining part of the carrier sheet 11 connected to the second cut 11A2, in the thickness direction of the second cut 11A2 may satisfy the following formula:

0≤TR2≤¾T

Since the skin patch sheet 10 makes it possible to peel the carrier sheet 11 from the substrate 13A from both sides of the patch tape 13 along the cut 11A, it is easy to peel the carrier sheet 11.

In the eleventh modification shown in FIG. 14, the outer shape of the carrier sheet 11 is rectangular. In the carrier sheet 11, a first region 11R1 surrounds a second region 11R2. The second region 11R2 has a rectangular shape, and the first region 11R1 has a frame shape that surrounds the second region 11R2. Each of the cuts 11A1 and 11A2 extends along the lengthwise direction. The base ends 11A1P and 11A2P of the respective cuts 11A1 and 11A2 are located on the same straight line extending along the lengthwise direction. The tips 11A1D and 11A2D of the respective cuts 11A1 and 11A2 are located within the second region 11R2. In the lengthwise direction, the tip 11A1D of the first cut 11A1 is apart from the tip 11A2D of the second cut 11A2.

The outer shape in each of the first to sixth modifications may be combined with the cuts 11A1 and 11A2 in each of the seventh to tenth modifications. In addition, the outer shape in each of the first to sixth modifications may be combined with the regions 11R1 and 11R2 and the cuts 11A1 and 11A2 in the eleventh modification.

### Configuration of Each Layer

The configuration of each layer provided in the skin patch sheet will be described below.

The carrier sheet 11 is made of a synthetic resin. The carrier sheet 11 is formed from, for example, a substrate sheet and a release layer. The release layer is laminated on the substrate sheet. When the carrier sheet 11 includes a substrate sheet and a release layer, the release layer is in contact with the substrate 13A of the patch tape 13. The substrate sheet may be made of, for example, a polypropylene (PP) resin or a polyethylene terephthalate (PET) resin. The substrate sheet may be any of a uniaxially stretched sheet, a biaxially stretched sheet, and a non-stretched sheet. The release layer may be made of, for example, a silicone resin.

The carrier sheet 11 may consist only of the substrate sheet described above. In this case, the surface of the substrate sheet in contact with the substrate 13A may be processed to facilitate the peeling of the substrate 13A from the substrate sheet. The processing on the contact surface of the substrate sheet may be, for example, embossing.

The carrier sheet 11 may have a thickness of 10 μm or more and 400 μm or less, as described above.

The release sheet 12 is configured so that the peeling strength between the release sheet 12 and the adhesive layer 13B is less than the peeling strength between the carrier sheet 11 and the substrate 13A. The release sheet 12 is made of a synthetic resin. The release sheet 12 is formed from, for example, a substrate sheet and a release layer. The release layer is laminated on the substrate sheet. When the release sheet 12 includes a substrate sheet and a release layer, the release layer is in contact with the adhesive layer 13B of the patch tape 13. The substrate sheet may be made of, for example, a polyethylene terephthalate (PET) resin. The substrate sheet may be any of a uniaxially stretched sheet, a biaxially stretched sheet, and a non-stretched sheet. The release layer may be made of, for example, a silicone resin.

The release sheet 12 may consist only of the substrate sheet described above. In this case, the surface of the substrate sheet in contact with the adhesive layer 13B may be processed to facilitate the peeling of the adhesive layer 13B from the substrate sheet. The processing on the contact surface of the substrate sheet may be, for example, embossing.

The release sheet 12 may have a thickness of, for example, 12 μm or more and 350 μm or less.

The substrate 13A is made of a synthetic resin. The synthetic resin for forming the substrate 13A may be, for example, a polyurethane resin. Accordingly, a substrate 13A having high adhesion suitability can be provided. The substrate 13A may have a thickness of, for example, 5 μm or more and 30 μm or less. The substrate 13A made of a polyurethane resin and having a small thickness is easily stretched even when a small amount of external force is applied to the substrate 13A to stretch the substrate 13A. Therefore, the substrate 13A has high conformability to the shape of the attachment target to which the patch tape 13 is attached, and can have high adhesion to the attachment target.

On the other hand, the substrate 13A, which has high conformability to the shape of the attachment target, also has high conformability to the carrier sheet 11, which covers the substrate 13A. Therefore, when the force to peel the carrier sheet 11 from the substrate 13A acts on the carrier sheet 11, the substrate 13A easily deforms according to the deformation of the carrier sheet 11.

The tensile elongation at break of the substrate 13A may be, for example, 130% or more. The tensile elongation at break can be determined according to JIS K 7161-1:2014 (ISO 527-1) “Plastics—Determination of tensile properties—Part 1: General principles” and JIS K 7127:1999 (ISO 527-3) “Plastics—Determination of tensile properties—Part 3: Test conditions for films and sheets.” The tensile elongation at break can be calculated by using a tensile strain at break when an object to be measured does not have a yield point, and a nominal tensile strain at break when an object to be measured has a yield point.

The tensile strength at 100% elongation of the substrate 13A, may be, for example, 4 N/cm or less. The tensile strength at 100% elongation is a value obtained by dividing the magnitude of the force measured when the strain defined in JIS K 7161-1:2014 (ISO 527-1) “Plastics—Determination of tensile properties—Part 1: General principles” reaches a predetermined value (100%) by the width of the test piece. The tensile strength at 100% elongation (T) (N/cm) can be calculated by the following formula.

T=F/W
where F is a magnitude (N) of the measured force, and W is the width (cm) of the test piece.

The substrate 13A may be made of a synthetic resin other than polyurethane resins. Examples of the synthetic resin other than polyurethane resins include polyvinylidene fluoride resin, ethylene-vinyl acetate copolymer resin, polypropylene resin, and polyethylene terephthalate resin.

The adhesive layer 13B is made of a synthetic resin, as with the substrate 13A. The synthetic resin for forming the adhesive layer 13B may be, for example, a polyurethane resin. The adhesive layer 13B may have a thickness of, for example, 5 μm or more and 25 μm or less.

At least one of the substrate 13A and the adhesive layer 13B may contain an active ingredient for the skin. That is, only the substrate 13A may contain an active ingredient, or only the adhesive layer 13B may contain an active ingredient, or the substrate 13A and the adhesive layer 13B both may contain an active ingredient. The active ingredient may be, for example, a cosmetic component, a beauty component, a pharmaceutical component, or the like.

### EXAMPLES

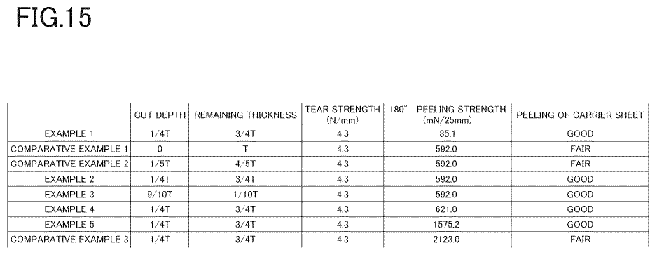

Examples and Comparative Example will be described with reference to FIG. 15.

### Example 1

A biaxially oriented polypropylene (OPP) sheet (manufactured by Futamura Chemical Co., Ltd., FOR-MP, thickness: 40 μm) having a matte surface as one of a pair of opposing surfaces was prepared as a carrier sheet. Then, an aqueous polyurethane containing ether-based polyol (manufactured by Mitsui Chemicals, Inc., TAKELAC WS-6021) (TAKELAC is a registered trademark) was applied to the matte surface of the OPP film, after which the aqueous polyurethane was dried to form a precursor layer. Subsequently, the precursor layer was left at room temperature for aging to thereby obtain a substrate having a thickness of 15 μm.

A release film composed of a polyethylene terephthalate (PET) sheet and a release layer made of a silicone resin (Toray Advanced Film Co., Ltd., Cerapeel WZ, thickness: 75 μm) (Cerapeel is a registered trademark) was prepared as a release sheet. Next, a urethane-based adhesive (manufactured by Toyochem Co., Ltd., SP-205) as a base resin, and a curing agent (manufactured by Toyochem Co., Ltd., T-501B) were prepared. After the curing agent was added to the base resin, the base resin containing the curing agent was stirred to obtain a coating liquid for forming an adhesive layer. The coating liquid was applied to the release film, and the coating liquid was then dried to obtain an adhesive layer having a thickness of 15 μm.

Then, the substrate and the adhesive layer were bonded together, thereby obtaining a laminate in which the OPP sheet, a patch tape, and the release sheet were laminated in this order. Then, the laminate was subjected to half-cut processing to cut out a region with a rectangular shape penetrating only the release film and the patch tape. Subsequently, portions of the release film and the patch tape outside the area that had been subjected to half-cut processing were removed from the OPP sheet. As a result, a release sheet and a patch tape each having a length of 50 mm in the lengthwise direction and a length of 10 mm in the widthwise direction, and an OPP sheet having a length of 50 mm in the lengthwise direction and a length of 20 mm in the widthwise direction were obtained.

In the OPP sheet, of the sides with a length of 50 mm extending along the lengthwise direction, two cuts were formed extending along the widthwise direction from a first side that did not overlap the patch tape to a second side that overlapped the patch tape. As a result, two cuts each having a straight line and having a length of 20 mm were formed. At this time, two cuts each having a depth of ¼T relative to the thickness T of the OPP sheet were formed, and the distance between the cuts in the lengthwise direction was set to 4 mm. Thus, a skin patch sheet of Example 1 was obtained.

### Comparative Example 1

A skin patch sheet of Comparative Example 1 was obtained in the same manner as in Example 1, except that no cuts were formed, and the skin patch sheet cut from the laminate was allowed to stand in an environment of 60 °C and a relative humidity of 90% for 5 days.

### Comparative Example 2

A skin patch sheet of Comparative Example 2 was obtained in the same manner as in Example 1, except that the depth of the cuts was changed to ⅕T, and the laminate after the cuts were formed was allowed to stand in an environment of 60 °C and a relative humidity of 90% for 5 days.

### Example 2

A skin patch sheet of Example 2 was obtained in the same manner as in Example 1, except that the laminate after the cuts were formed was allowed to stand in an environment of 60 °C and a relative humidity of 90% for 5 days.

### Example 3

A skin patch sheet of Example 3 was obtained in the same manner as in Example 1, except that the depth of the cuts was changed to 9/10T, and the laminate after the cuts were formed was allowed to stand in an environment of 60 °C and a relative humidity of 90% for 5 days.

### Example 4

A skin patch sheet of Example 4 was obtained in the same manner as in Example 1, except that 40% by weight of concentrated glycerin (manufactured by Kanto Chemical Co., Inc., 17029-08, quasi-drug raw material standard) based on the solids content of the base resin was mixed with the coating liquid for forming an adhesive layer.

### Example 5

A skin patch sheet of Example 5 was obtained in the same manner as in Example 1, except that the laminate after the cuts were formed was allowed to stand in an environment of 60 °C and a relative humidity of 90% for 10 days.

### Comparative Example 3

A skin patch sheet of Comparative Example 3 was obtained in the same manner as in Example 1, except that the laminate after the cuts were formed was allowed to stand in an environment of 60 °C and a relative humidity of 90% for 15 days.

### Evaluation Method

### Trouser Tear Strength

The tear strength of the carrier sheet was measured by a method according to JIS K 7128-1 (1998) “Plastics—Film and sheeting—Determination of tear resistance—Part 1: Trouser tear method.” At this time, a test piece with a width of 50 mm and a length of 150 mm was prepared from each of the carrier sheets of the Examples and Comparative Examples. Then, in one end portion of the test piece in the length direction, a notch starting from the center of the width direction and extending along the length direction was formed in the test piece, whereby a first piece portion and a second piece portion separated by the notch were formed in the test piece. At this time, the length of the notch was set to 75 mm.

### 180° Peeling Strength

The peeling strength of the substrate relative to the carrier sheet was measured by a method according to JIS Z 0237:2009 “Testing methods of pressure-sensitive adhesive tapes and sheets.” In the measurement of the peeling strength, a universal tester (manufactured by Shimadzu Corporation, Autograph AGS-X, load cell 5 kN) was used. When measuring the peeling strength, a test piece with a width of 25 mm was cut out from each of the skin patch sheets of the Examples and Comparative Examples. After the substrate was fixed to the universal tester, the strength when peeling the carrier sheet at 180° with respect to the substrate was calculated. Then, the universal tester was used to pull the first piece portion and the second piece portion in opposite directions, and the tear strength (N/mm) when the tear amount reached 50 mm was measured.

### Tearability

Regarding the skin patch sheets of the Examples and Comparative Examples, the carrier sheet was torn from a portion of each cut located in the first region of the carrier sheet. That is, the skin patch sheets of the Examples and Comparative Examples were evaluated for peeling of the carrier sheet. The tearability at this time was evaluated with the following criteria. The tearability of the skin patch sheets of the Examples and Comparative Examples was evaluated 5 times.

+: Starting from the cuts, the carrier sheet could be torn across the entire widthwise direction. Accordingly, the carrier sheet could be smoothly peeled from the substrate.

−: Starting from the cuts, the carrier sheet could be torn across the entire widthwise direction; however, the resistance when tearing the carrier sheet was high.

### Evaluation Results

The evaluation results of the trouser tear strength, 180° peeling strength, and tearability were as shown in FIG. 15.

As shown in FIG. 15, it was recognized that the trouser tear strength was 4.3 N/mm in the carrier sheets of the skin patch sheets of Examples 1 to 5 and Comparative Examples 1 to 3.

It was recognized that the 180° peeling strength was 85.1 mN/25 mm in Example 1, and 592.0 mN/25 mm in Examples 2 and 3 and Comparative Examples 1 and 2. It was recognized that the 180° peeling strength was 621.0 mN/25 mm in Example 4, 1575.2 mN/25 mm in Example 5, and 2123.0 mN/25 mm in Comparative Example 3.

It was recognized that the evaluation of tearability was + in Examples 1 to 5 and − in Comparative Examples 1 to 3. Specifically, since the depth of each cut was shallow in Comparative Examples 1 and 2, external force was required to tear the remaining part of the carrier sheet, thereby confirming that the tearability of the carrier sheet was low. Further, since the adhesion strength between the carrier sheet and the substrate was high in Comparative Example 3, when external force for peeling the carrier sheet from the substrate was applied to the carrier sheet, the patch tape lifted together with the carrier sheet, thereby confirming that the tearability of the carrier sheet was low.

It was also recognized that among Examples 1, 2, and 4, the resistance when tearing the carrier sheet was lowest in Example 1, in which the 180° peeling strength was lowest, whereas the resistance when tearing the carrier sheet was highest in Example 4, in which the 180° peeling strength was highest. It was also recognized that in Examples 2 and 3 and Comparative Examples 1 and 2, the lower the depth of the cuts, the lower the resistance when tearing the carrier sheet.

As described above, the skin patch sheet according to an embodiment achieves the following advantageous effects.
(1) In the carrier sheet 11 having a thickness of 10 μm or more and 400 μm or less, the 180° peeling strength is 2 N/25 mm or less, and the depth of the cut 11A extending from the outside of the patch tape 13 to the inside of the patch tape 13 is included within the range of ¼T or more and T or less. Therefore, it is easy to peel the carrier sheet 11 from substrate 13A starting from the cut 11A.
(2) In the carrier sheet 11 that has a trouser tear strength of 10 N/mm or less, the 180° peeling strength is 2 N/mm or less, and the remaining thickness TR1 of the carrier sheet 11 in the cut 11A extending from the outside of the patch tape 13 to the inside of the patch tape 13 is included within the range of 0 or more and ¾T or less. Therefore, it is easy to peel the carrier sheet 11 from substrate 13A starting from the cut 11A.
(3) A portion of the cut 11A located on the patch tape 13 may not penetrate the carrier sheet 11. In this case, during the production of the skin patch sheet 10, the carrier sheet and the substrate can be prevented from being cut, and foreign substances can be prevented from reaching the substrate through the cut 11A before using the skin patch sheet.
(4) When the trouser tear strength is 10 N/mm or less, it is possible to increase the tearability of the carrier sheet 11. Moreover, during the production of the skin patch sheet 10, the carrier sheet 11 and the substrate 13A can be prevented from being cut, and foreign substances can be prevented from reaching the substrate 13A through the cut 11A before using the skin patch sheet 10.
(5) When the base ends 11A1P and 11A2P of the respective cuts 11A1 and 11A2 face each other across the patch tape 13, it is possible to peel the carrier sheet 11 from the substrate 13A from the both sides of the patch tape 13 along the cuts 11A1 and 11A2. Therefore, it is easy to peel the carrier sheet 11.
(6) When the skin patch sheet 10 includes a pinch portion 11N defined by the cuts 11A1 and 11A2, it is possible for the user of the skin patch sheet 10 to hold the pinch portion 11N of the carrier sheet 11. Therefore, it is easy to apply external force for peeling the carrier sheet 11 from the substrate 13A to the carrier sheet 11.

1. A skin patch sheet comprising a carrier sheet, a release sheet, and a patch tape located between the carrier sheet and the release sheet,
the patch tape comprising a substrate that is in contact with the carrier sheet and has higher tensile elongation at break than that of the carrier sheet and the release sheet, and an adhesive layer that is in contact with the release sheet and is to be attached to the skin after the release sheet is peeled from the adhesive layer,
wherein the area of the carrier sheet is larger than the area of the patch tape, and the carrier sheet includes a first region that does not overlap the patch tape and a second region that overlaps the patch tape,
the carrier sheet has a cut extending beyond a boundary between the first region and the second region from a portion of the outer edge of the carrier sheet included in the first region toward the patch tape when viewed from a perspective facing a plane on which the carrier sheet extends,
a 180° peeling strength according to JIS Z 0237:2009 between the carrier sheet and the substrate is 2 N/25 mm or less,
the carrier sheet has a thickness of 10 μm or more and 400 μm or less, and
when the thickness of the carrier sheet is T, a depth D1 of the cut satisfies the following formula: ¼T≤D1≤T.

2. The skin patch sheet according to claim 1, wherein ¼T≤D1≤ 9/10T.

3. The skin patch sheet according to claim 1, wherein a depth of the cut in a portion that overlaps the patch tape in the carrier sheet is shallower than a depth of the cut in a portion that does not overlap the patch tape in the carrier sheet.

4. The skin patch sheet according to claim 1, wherein a depth of the cut in a portion that overlaps the patch tape in the carrier sheet is shallower than a depth of the cut in a portion that does not overlap the patch tape in the carrier sheet, and
the carrier sheet has a trouser tear strength according to JIS K 7128-1:1998 of 10 N/mm or less.

5. The skin patch sheet according to claim 1, wherein the cut is a first cut,
the carrier sheet further comprises a second cut extending beyond a boundary between the first region and the second region from a portion of the outer edge of the carrier sheet included in the first region toward the patch tape when viewed from a perspective facing a plane on which the carrier sheet extends,
when the thickness of the carrier sheet is T, a depth D2 of the second cut satisfies the following formula: ¼T≤D2≤T, and
base ends of the cuts located at the outer edge of the carrier sheet face each other across the patch tape.

6. The skin patch sheet according to claim 1, wherein the cut is a first cut,
the carrier sheet further comprises a second cut extending beyond a boundary between the first region and the second region from a portion of the outer edge of the carrier sheet included in the first region toward the patch tape when viewed from a perspective facing a plane on which the carrier sheet extends,
when the thickness of the carrier sheet is T, a depth D2 of the second cut satisfies the following formula: ¼T≤D2≤T,
when viewed from a perspective facing a plane on which the carrier sheet extends, the first cut and the second cut are spaced along a first direction, and each cut extends along a second direction that intersects with the first direction, and
the carrier sheet comprises a pinch portion that is sandwiched between the first cut and the second cut, and extends along the second direction.

7. The skin patch sheet according to claim 1, wherein the substrate or the adhesive layer contains an active ingredient for the skin.

8. The skin patch sheet according to claim 1, wherein the carrier sheet includes a substrate sheet and the substrate sheet is made of a polypropylene resin or a polyethylene terephthalate resin.

9. The skin patch sheet according to claim 8, wherein the carrier sheet further includes a release layer, the release layer is in contact with the substrate of the patch tape.

10. The skin patch sheet according to claim 1, wherein the substrate has a thickness of 5 μm or more and 30 μm or less and the tensile elongation at break of 130% or more.

11. A skin patch sheet comprising a carrier sheet, a release sheet, and a patch tape located between the carrier sheet and the release sheet,
the patch tape comprising a substrate that is in contact with the carrier sheet and has higher tensile elongation at break than that of the carrier sheet and the release sheet, and an adhesive layer that is in contact with the release sheet and is to be attached to the skin after the release sheet is peeled from the adhesive layer,
wherein the area of the carrier sheet is larger than the area of the patch tape, and the carrier sheet includes a first region that does not overlap the patch tape and a second region that overlaps the patch tape,
the carrier sheet has a cut extending beyond a boundary between the first region and the second region from a portion of the outer edge of the carrier sheet included in the first region toward the patch tape when viewed from a perspective facing a plane on which the carrier sheet extends,
a 180° peeling strength according to JIS Z 0237:2009 between the carrier sheet and the substrate is 2 N/25 mm or less,
the carrier sheet has a trouser tear strength according to JIS K 7128-1:1998 of 10 N/mm or less, and
when the thickness of the carrier sheet is T, a remaining thickness TR1 of the carrier sheet connected to the cut in a thickness direction of the carrier sheet satisfies the following formula: 0≤TR1≤¾T.

12. The skin patch sheet according to claim 11, wherein 1/10T≤TR1≤¾T.

13. The skin patch sheet according to claim 12, wherein a depth of the cut in a portion that overlaps the patch tape in the carrier sheet is shallower than a depth of the cut in a portion that does not overlap the patch tape in the carrier sheet.

14. The skin patch sheet according to claim 11, wherein a depth of the cut in a portion that overlaps the patch tape in the carrier sheet is shallower than a depth of the cut in a portion that does not overlap the patch tape in the carrier sheet, and
the carrier sheet has a thickness of 10 μm or more and 400 μm or less.

15. The skin patch sheet according to claim 11, wherein the cut is a first cut,
the carrier sheet further comprises a second cut extending beyond a boundary between the first region and the second region from a portion of the outer edge of the carrier sheet included in the first region toward the patch tape when viewed from a perspective facing a plane on which the carrier sheet extends,
when the thickness of the carrier sheet is T, a depth D2 of the second cut satisfies the following formula: ¼T≤D2≤T, and
base ends of the cuts located at the outer edge of the carrier sheet face each other across the patch tape.

16. The skin patch sheet according to claim 11, wherein the cut is a first cut,
the carrier sheet further comprises a second cut extending beyond a boundary between the first region and the second region from a portion of the outer edge of the carrier sheet included in the first region toward the patch tape when viewed from a perspective facing a plane on which the carrier sheet extends,
when the thickness of the carrier sheet is T, a depth D2 of the second cut satisfies the following formula: ¼T≤D2≤T,
when viewed from a perspective facing a plane on which the carrier sheet extends, the first cut and the second cut are spaced along a first direction, and each cut extends along a second direction that intersects with the first direction, and
the carrier sheet comprises a pinch portion that is sandwiched between the first cut and the second cut, and extends along the second direction.

17. The skin patch sheet according to claim 1, wherein the substrate or the adhesive layer contains an active ingredient for the skin.

18. The skin patch sheet according to claim 11, wherein the carrier sheet includes a substrate sheet and the substrate sheet is made of a polypropylene resin or a polyethylene terephthalate resin.

19. The skin patch sheet according to claim 8, wherein the carrier sheet further includes a release layer, the release layer is in contact with the substrate of the patch tape.

20. The skin patch sheet according to claim 1, wherein the substrate has a thickness of 5 μm or more and 30 μm or less and the tensile elongation at break of 130% or more.
